(19) United States
(12) Plant Patent Application Publication
(10) Pub. No.: US 2003/0041360 P1
(43) Pub. Date: Feb. 27, 2003

(54) Climbing rose plant named 'Meivaleir'

(76) Inventor: Alain A. Meilland, Antibes (FR)

Correspondence Address: BURNS, DOANE, SWECKER & MATHIS, L.L.P., Post Office Box 1404, Alexandria, VA, 22313-1404, US

(21) Appl. No.: 09/933,174

(22) Filed: Aug. 21, 2001

(51) Int. Cl. A01H 5/00

(52) U.S. Cl. PLT/111

(57) ABSTRACT

A new and distinct variety of rose plant is provided which abundantly forms attractive double blossoms having a luminous yellow-orange coloration. The plant exhibits a climbing growth habit, dense medium green semi-glossy foliage, and good vigor. The attractive medium green foliage contrasts nicely with the light-colored blossoms. The new variety is particularly well suited for growing as attractive ornamentation in the landscape such as in parks and gardens.

### SUMMARY OF THE INVENTION

The new variety of Rosa hybrida Climbing rose plant was created by artificial pollination wherein two parents were crossed which previously had been studied in the hope that they would contribute the desired characteristics. The female parent (i.e., the seed parent) of the ‘Meipalsar’ variety (non-patented in the United States). The male parent (i.e., the pollen parent) was the product of the pollination of the ‘Korwest’ variety (non-patented in the United States) and the ‘Circus’ variety (U.S. Plant Pat. No. 2,074). The parentage of the new variety can be summarized as follows:

(‘Meipalsar’×(‘Korwest’×‘Circus’).

The seeds resulting from the above pollination were sown and small plants were obtained which were physically and biologically different from each other. Selective study resulted in the identification of a single plant of the new variety.

It was found that the new variety of rose plant of the present invention possesses the following combination of characteristics:

(a) exhibits a climbing growth habit,

(b) abundantly forms attractive double blossoms having a luminous yellow-orange coloration,

(c) forms dense medium green semi-glossy foliage that contrasts well with the light-colored blossoms,

(d) exhibits good vigor, and

(e) is particularly well suited for growing as attractive ornamentation in the landscape.

The new variety well meets the needs of the horticultural industry and can be grown to advantage in parks and gardens.

The new variety has been found to undergo asexual propagation in France by a number of routes, including budding, grafting, and cuttage. Asexual propagation by the above-mentioned techniques in France has shown that the characteristics of the new variety are stable and are strictly transmissible by such asexual propagation from one generation to another.

The new variety has been named the ‘Meivaleir’ variety.

### BRIEF DESCRIPTION OF THE PHOTOGRAPH

The accompanying photograph shows as nearly true as it is reasonably possible to make the same, in a color illustration of this character, typical specimens of the plant parts of the new variety. The rose plants of the new variety were two years of age and were observed during June while budded on Rosa froebelli understock and growing in outdoors at Le Cannet des Maures, Var, France. Dimensions in centimeters are indicated at the bottom of the photograph.

FIG. 1—illustrates a specimen of a young shoot;

FIG. 2—illustrates a specimen of a floral bud before the opening of the sepals;

FIG. 3—illustrates a specimen of a floral bud at the opening of the sepals;

FIG. 4—illustrates a specimen of a floral bud at the opening of the petals;

FIG. 5—illustrates a specimen of a flower in the course of opening;

FIG. 6—illustrates a specimen of an open flower—plan view—obverse;

FIG. 7—illustrates a specimen of an open flower—plan view—reverse;

FIG. 8—illustrates a specimen of a fully open flower—plan view—obverse;

FIG. 9—illustrates a specimen of a fully open flower—plan view—reverse;

FIG. 10—illustrates a specimen of a floral receptacle showing the arrangement of the stamens and pistils;

FIG. 11—illustrates a specimen of a floral receptacle showing the arrangement of the pistils (stamens removed);

FIG. 12—illustrates a specimen of a flowering stem;

FIG. 13—illustrates a specimen of a main branch;

FIG. 14—illustrates a specimen of a leaf with three leaflets plan view—upper surface;

FIG. 15—illustrates a specimen of a leaf with five leaflets plan view—under surface; and

FIG. 16—illustrates a specimen of a leaf with seven leaflets plan view—upper surface.

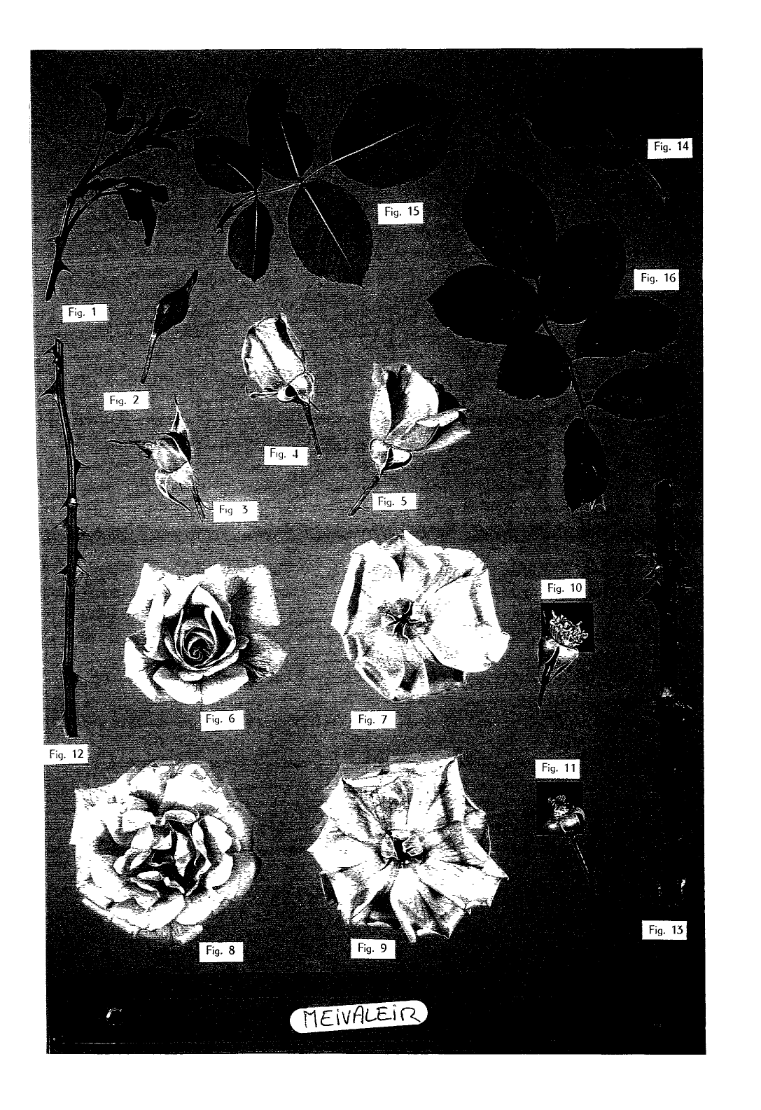

### DETAILED DESCRIPTION

The chart used in the identification of the colors is that of The Royal Horticultural Society (R.H.S. Colour Chart). The description is based on the observation of two year-old plants during June while budded on Rosa froebelli understock and growing outdoors at Le Cannet des Maures, Var, France. The coloration in more common terms precedes reference to the chart in some instances. Such terminology is to be accorded its ordinary dictionary significance.

Class: Climbing.

Plant:

Height.—approximately 150 to 170 cm at the end of the growing season.

Habit.—climbing.

Branches:

Color.—young stems: near Yellow-Green Group 146C.—adult wood: near Yellow-Green Group 146A and 146B.

Thorns.—size: medium (as illustrated).—quantity: moderately numerous (as illustrated).—color: near Greyed-Orange Group 164B at the base and near Greyed-Orange Group 164A and 164B at the tip.—configuration: deeply concave on the lower surface.

Leaves:

Stipules.—adnate, pectinate, and rather broad.

Petioles.—near Yellow-Green Group 146B on its upper surface and near Yellow-Green Group 153D on the under surface.

Leaflets.—number: 3, 5, and 7.—shape: the base is generally symmetrical and obtuse and the tip is generally symmetrical with a broad point (as illustrated). —serration: regular (as illustrated). —texture: consistent, and rather firm. —general appearance: very dense, medium green, and semi-glossy foliage. —color (young foliage): upper surface: near Green Group 139A. under surface: near Yellow-Green Group 147C. —color (adult foliage): upper surface: near Green Group 139A. under surface: near Yellow-Green Group 147C.

Inflorescence:

Number of flowers.—usually one or two flowers per stem.

Peduncle.—near Yellow-Green Group 146D in coloration, and medium in size with aciculae and prickles.

Sepals.—upper surface: elongated, tomentose, and near Greyed-Green Group 194B. —under surface: near Yellow-Green Group 146B with slight anthocyanin coloration and commonly with only a few short extensions (as illustrated).

Buds.—shape: conical. —length: approximately 2.5 cm on average. —color: upper surface: Chrome Yellow, near Yellow-Orange Group 14C, and lightly suffused with Empire Rose, Red Group 48C. under surface: Chrome Yellow, near Yellow-Orange Group 14C, and lightly suffused with Empire Rose, Red Group 48C.

Flower.—shape: double and cup-shaped. —diameter: approximately 9 to 10 cm on average. —color (when opening begins): upper surface: Chrome Yellow, near Yellow-Orange Group 14C, and lightly suffused with Empire Rose, Red Group 48C. under surface: Chrome Yellow, near Yellow-Orange Group 14C, and lightly suffused with Empire Rose, Red Group 48C. —color (when blooming): upper surface: Chrome Yellow, near Yellow-Orange Group 14C, and lightly suffused with Empire Rose, Red Group 48C. under surface: Chrome Yellow, near Yellow-Orange Group 14C, and lightly suffused with Empire Rose, Red Group 48C. —color (at end of blooming): upper surface: Chrome Yellow, near Yellow-Orange Group 14C, and lightly suffused with Empire Rose, Red Group 48C. under surface: Chrome Yellow, near Yellow-Orange Group 14C, and lightly suffused with Empire Rose, Red Group 48C. —petal number: approximately 25 to 39 on average. —fragrance: none. —lasting quality: long on the plant. —petal shape: with a cordate base and a reflexed tip. —receptacle: near Yellow-Green Group 146D in coloration, and funnel-shaped in longitudinal section.

Development:

Vegetation.—very vigorous and strong.

Blooming.—abundant.

Resistance to diseases.—very good.

I claim:

1. A new and distinct variety of rose plant characterized by the following combination of characteristics:
(a) exhibits a climbing growth habit,
(b) abundantly forms attractive double blossoms having a luminous yellow-orange coloration,
(c) forms dense medium green semi-glossy foliage that contrasts well with the light-colored blossoms,
(d) exhibits good vigor, and
(e) is particularly well suited for growing as attractive ornamentation in the landscape;
substantially as herein shown and described.
